(19) United States
(12) Patent Application Publication
(10) Pub. No.: US 2010/0016746 A1
(43) Pub. Date: Jan. 21, 2010

(54) PERSONAL ALERTING DEVICE FOR USE WITH DIAGNOSTIC DEVICE

(76) Inventors: David R. Hampton, Woodinville, WA (US); Forrest C.M. Pape, New Brighton, MN (US); Paul G. Krause, Shoreview, MN (US); Dwight H. Warkentin, Ardin Hills, MN (US)

Correspondence Address: MEDTRONIC, INC., 710 MEDTRONIC PARKWAY NE, MINNEAPOLIS, MN, 55432-9924, US

(21) Appl. No.: 12/173,778

(22) Filed: Jul. 15, 2008

(51) Int. Cl. A61B 5/0402

(52) U.S. Cl. 600/523

(57) ABSTRACT

A personal altering device and method of generating an alert of an event detected by an implanted diagnostic device are provided. The method can include transmitting an event signal to the personal alerting device when one or more of the following occurs: ischemia is detected, a deviation from the baseline waveform is detected, a life-threatening trend begins, or symptoms are indicated. The method can also include generating an alarm signal from the personal alerting device, and the alarm signal can include one or more of a visual message, a light, a vibration, and a sound. In addition, the method can include prompting further action to be taken.

### TECHNICAL FIELD

This disclosure relates generally to personal alerting devices and more particularly to personal alerting devices for use with implanted or subcutaneous diagnostic devices.

### BACKGROUND

Implantable devices have been known that provide communication to a patient using vibrations or sound. However, most conventional implantable devices are unable to signal their findings to a patient or to medical personnel without an accompanying device to receive, display, and relay the signals.

### SUMMARY

In one or more embodiments, a method is provided of generating an alert of an event detected by an implanted diagnostic device. The method includes providing a personal alerting device. The method includes transmitting an event signal to the personal alerting device from the implanted diagnostic device when, for example, any of the following occurs: ischemia is detected, a deviation from the baseline electrocardiogram waveform is detected, a life-threatening trend begins, or symptoms are indicated. The method can also include storing the event signal in the personal alerting device, and the event signal can include an event onset timestamp, an event type, and an event electrocardiogram waveform. The method can further include generating an alarm signal from the personal alerting device, and the alarm signal can include one or more of a visual message, a light, a vibration, a sound, and a shock. Finally, the method can include prompting further action to be taken through the personal alerting device.

In one or more embodiments, a personal alerting system can be provided that generates an alert of an event. The personal alerting system can include an implanted diagnostic device that transmits an event signal. The personal alerting system can include a personal alerting device that receives the event signal. The personal alerting device can include a processor, a communication module, and memory. The personal alerting device can also include one or more of a speaker, an indicator light, and a vibration generator. In addition, the personal alerting device can include a display to indicate an alarm signal and prompt further action to be taken.

### DRAWINGS

The above-mentioned features and objects of the present disclosure will become more apparent with reference to the following description taken in conjunction with the accompanying drawings wherein like reference numerals denote like elements and in which:

FIG. 1 is a schematic diagram of a personal alerting device in communication with an implanted diagnostic device in accordance with an embodiment of the present disclosure.

FIG. 2 is a block diagram illustrating the various components of one embodiment of the personal alerting device configured to operate in accordance with the present disclosure.

FIG. 3 is a block diagram illustrating the various components of another embodiment of the personal alerting device and a subcutaneous monitoring device configured to operate in accordance with the present disclosure.

### DETAILED DESCRIPTION

The present disclosure describes a personal alerting device that can be worn or carried by a patient in order to communicate information from the patient's implanted diagnostic device, including combination diagnostic/therapeutic devices, diagnostic device to the patient and/or to potential rescuers when life-threatening events or trends are detected, such as acute myocardial infarction, ischemia, or non-contractile myocardium.

In the following description, numerous embodiments are set forth in order to provide a thorough understanding of the invention. It will be apparent, however, to one skilled in the art, that these and other embodiments may be practiced without these specific details. In some instances, features well-known to those skilled in the art have not been described in detail in order not to obscure the present disclosure.

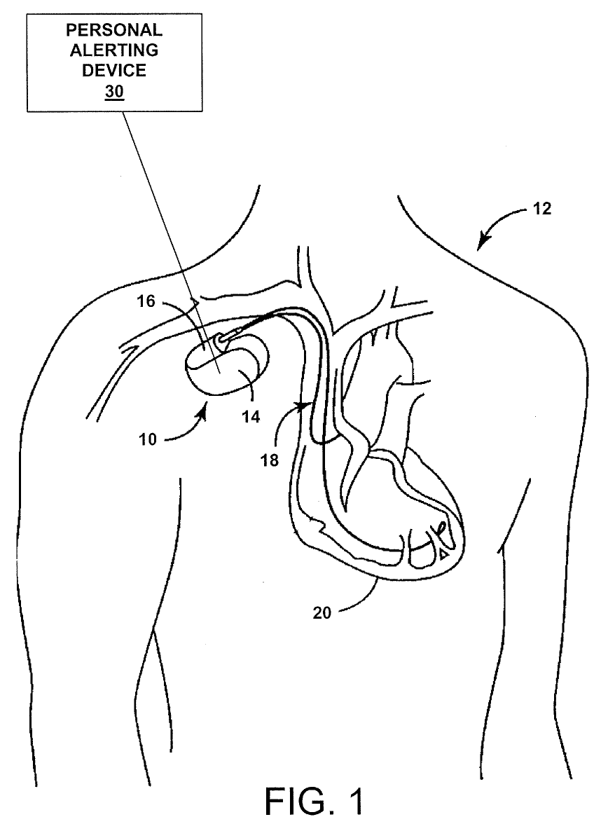

FIG. 1 is a simplified schematic view of an implanted diagnostic device 10 that is implanted in a human body 12. The implanted diagnostic device 10 can include a hermetically sealed enclosure 14 and connector module 16 for coupling the implanted diagnostic device 10 to electrical leads 18 arranged within the body 12, such as pacing and sensing leads 18 connected to portions of the heart 20 for delivery of pacing pulses to a patient's heart 20 and sensing of the heart 20 conditions. While the implanted diagnostic device 10 is depicted in a pacemaker configuration in FIG. 1, the implanted diagnostic device 10 can comprise any suitable type of implanted device including, but not limited to, an implanted subcutaneous diagnostic device (e.g., a device the size of a fingertip that slips under the patient's skin), an implanted submuscular diagnostic device, an implantable cardioverter-defibrillator or an implantable combination pacemaker-cardioverter-defibrillator, as well as embodiments including implantable brain stimulators, implantable loop recorders, implantable gastric system stimulators, implantable nerve stimulators or muscle stimulators, implantable lower colon stimulators, implantable drug or beneficial agent dispensers or pumps, implantable cardiac signal loops or other types of recorders or monitors, implantable gene therapy delivery devices, implantable incontinence prevention or monitoring devices, or implantable insulin pumps or monitoring devices.

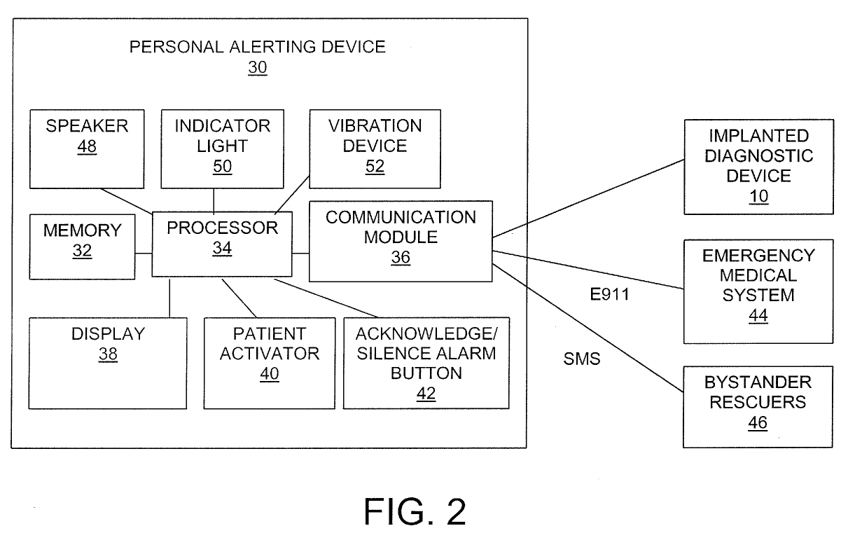

In some embodiments, the implanted diagnostic device 10 can communicate with a personal alerting device 30. The implanted diagnostic device 10 can monitor life-sustaining physiologic cardiorespiratory, renal, or pulmonary functions and can communicate information via the personal alerting device 30 to the patient and to potential rescuers, a monitoring service, or other monitoring technology when life-threatening trends or events are detected. Some embodiments of the invention provide a dedicated personal alerting device 30 that can be worn or carried by the patient. FIG. 2 illustrates a simplified block diagram of the personal alerting device 30 according to some embodiments. The personal alerting device 30 can include memory 32, a processor 34, a communication module 36, and a display 38. In some embodiments, the personal alerting device 30 can also include a patient activator 40 and/or an acknowledge/silence alarm button 42. The personal alerting device 30 can communicate with the implanted diagnostic device 10, with the emergency medical system 44 (e.g., via an emergency 911 telephone system), and with bystander rescuers 46 (e.g., via the display 38 or short message service communication to cellular phones). The personal alerting device 30 can include a speaker 48, an indictor light 50, and/or a vibration device 52.

The personal alerting device 30 can receive via the communication module 36 transmitted event signals from the implanted diagnostic device 10 when an initiating event occurs. In some embodiments, the implanted diagnostic device 10 can detect ischemia as a change that matches predetermined universal diagnostic criteria for ischemia (which can be stored in the personal alerting device 30). In some embodiments, the personal alerting device 30 can receive event signals from the implanted diagnostic device 10 when threshold-criteria or trend-criteria are met. The implanted diagnostic device 10 can generate an event signal after detection of defined events and/or detection of significant trends toward an ischemic state in the patient's data. The implanted diagnostic device 10 can generate an event signal after detection of a characteristic deviation or other significant change from the patient's own baseline electrocardiogram waveform. The implanted diagnostic device 10 can monitor non-electrocardiogram signals, such as heart sounds, changes in tissue perfusion, impedance, etc. The implanted diagnostic device 10 can also generate an event signal when the patient indicates that symptoms are occurring that are indicative of an ischemic event (e.g., chest pain). To indicate that symptoms are occurring, the patient can press the patient activator 40 on the personal alerting device 30. In some embodiments, when the implanted diagnostic device 10 detects an event or the patient indicates that symptoms are occurring, one or more electrocardiograms or electrograms from one or more chest positions or other body locations can be recorded using electrodes coupled to the personal alerting device 30. Alternatively, the implanted diagnostic device 10 can record and send one or more electrocardiograms or electrograms or other measured physiological signals to the personal alerting device 30. In some embodiments, the implanted diagnostic device 10 can store an electrogram strip of the signal that triggered the acute myocardial infarction alarm for later review via an implantable device programmer or an implanted device monitor. In some embodiments, the implanted diagnostic device 10 or the personal alerting device 30 can provide secondary diagnostic capabilities that can be used to assess cardiorespiratory status when the patient indicates symptoms are present. For example, the personal alerting device 30 can include an embedded electrocardiogram monitor that can be used to assess the surface electrocardiogram for signs of new ST-elevation when a patient signals with the patient activator 40 that he is suffering chest pain. The ST elevations or variations shown on a surface electrocardiogram may be significantly different in appearance than the ST elevations or variations detected by the implantable diagnostic device 10. However, the personal alerting device 30 can be equipped to process these differences.

The personal alerting device 30 can store data in memory 32, such as the event onset timestamp, the event type, the event duration, amplitude, and/or the supporting evidence, such as event electrocardiograms or measured ST-deviation values, baseline electrocardiograms, and electrograms. The personal alerting device 30 can also store patient and medical information, such as contact information or a baseline electrocardiogram, which, in some embodiments, can be transmitted along with the alarm signal to the emergency medical system 44. The personal alerting device 30 can store historical-related, periodic-related, and/or episode-related data for subsequent transmission to the emergency medical system 44. For example, the personal alerting device 30 can store episode-related data for subsequent transmission to medical personal (e.g., emergency dispatch, the cardiac care unit, and the cardiac catherization lab). In some embodiments, the personal alerting device 30 can store the event signal for later printing, transmission, comparison, or retrieval. In one embodiment, the personal alerting device 30 can store a digitized 12-lead electrocardiogram (collected from a standard electrocardiogram recorder) so that emergency medical personnel can access the full electrocardiogram at the time of the patient's alarm signal and compare it to the patient's current electrocardiogram. In some embodiments, the personal alerting device 30 can also receive data via the communication module 36 from medical personnel in order to tailor the personal alerting device 30 for improved performance, for example, by modifying alerting thresholds or updating baseline waveforms for the specific patient.

In some embodiments, upon receiving the event signal from the implanted diagnostic device 10, the personal alerting device 30 can perform additional processing of the event signal before generating an alarm signal. For example, the implanted diagnostic device 10 can perform cursory processing and the personal alerting device 30 can have additional sensors and/or additional or more powerful computing capabilities. In one embodiment, the personal alerting device 30 can include a barometric pressure sensor and a blood pressure change detected by the implanted diagnostic device 10 can trigger an event signal. However, before generating the alarm signal, the personal alerting device 30 can to determine whether or not the blood pressure change was a dramatic barometric pressure change. In one embodiment, the implanted diagnostic device 10 can detect an ST segment change and trigger an event signal. However, before generating the alarm signal, the personal alerting device 30 can perform an additional signal-quality check on an uplinked electrogram (using more sophisticated digital signal processing capabilities in the personal alerting device 30 than in the implanted diagnostic device 10).

Upon receiving the event signal from the implanted diagnostic device 10, the personal alerting device 30 can generate a patient-perceptible or patient-accessible alarm signal, such as a flashing light, a vibrating alert, an audible tone, a smell, and/or an electrical shock. In some embodiments, the alarm signal can include a timer that automatically forwards data if the patient does not acknowledge the alarm signal within a set period. Some embodiments of the invention provide a personal alerting device 30 that can be worn by the patient as a bracelet, necklace, belt, wristwatch, an earring, body ornament, or similar accessory, and serves as a companion to the implanted diagnostic device 10. In other embodiments, the personal alerting device 30 can be a device similar in size to a pager, a Wi-Fi sniffer, a personal digital assistant, or a cellular phone, and the personal alerting device 30 may also incorporate the functionality of these devices. The alarm signals can be tailored to different forms, such as by being classified by alarm type and/or urgency, by escalating or de-escalating trends or event sequences, or by implanted device service alarms from the implanted diagnostic device 10 (such as a low battery in the implanted diagnostic device 10).

In some embodiments, the patient can acknowledge the alarm signal by pressing the acknowledge/silence alarm button or switch 42. In some embodiments, the personal alerting device 30 can include a voice-activated system that allows the patient to indicate that he is experiencing symptoms and to initiate a data collection from the implanted diagnostic device 10 and/or communicate with medical personnel. In some embodiments, the patient can log symptoms in the personal alerting device 30, such as chest pain scores and drug consumption (which can also be transmitted to emergency response personnel). The patient can acknowledge the event and can then take further action, for example, as directed by the display 38 of the personal alerting device 30.

In some embodiments, the personal alerting device 30 can automatically acknowledge or silence the alarm signal if the patient does not respond within a certain time period. Some embodiments of the personal alerting device 30 can also provide controls to perform one or more of the following functions: to take action if an alarm is not acknowledged by the patient, to automatically relay the alarm to the emergency medical system 44 or to bystander rescuers 46, and/or to automatically initiate enhanced recording or diagnostic functions in the implanted diagnostic device 10.

In some embodiments, the personal alerting device 30 can prompt the patient, the emergency medical system 44, or bystander rescuers 46 to take further action base on the nature, duration, and severity of the detected event. For example, the display 38 of the personal alerting device 30 can show information generated specifically for the patient (when the patient acknowledges the alarm signal), specifically for bystander rescuers 46 (when the patient does not acknowledge the alarm signal), and/or specifically for emergency medical technicians. In some embodiments, the personal alerting device 30 can stream and display a real-time electrogram on the display 38. In some embodiments, the personal alerting device 30 can display additional diagnostics, such as blood pressure, oxygen, cardiac output, and a measure of heart contractility. In some embodiments, the display 38 of the personal alerting device 30 can instruct the patient to self-administer drugs, such as aspirin or nitroglycerin.

In some embodiments, the personal alerting device 30 can automatically alert one or more of the following medical personnel: emergency responders, tele-monitoring services, dispatch services, designated caregivers (including family members, a primary caregiver, or a call center so that the cause of the alarm signal can be investigated. The personal alerting device 30 can forward the alarm signal and the accompanying data to a call center where the alarm signal can be screened by medical personnel to eliminate any false positive alarm signals. The medical personnel in the call center can call the patient and/or forward only the true positive alarm signals to the emergency medical system 44. The medical personnel in the call center can also contact a friend or relative of the patient to ensure that the patient is being taken to the nearest hospital.

The patient, taking note of the alarm signal, can take professionally-prescribed actions that can include one or more of the following actions: entering symptoms into a diary, performing further diagnostic tests, initiating therapeutic mitigations (such as providing pacing therapy to reduce stress on the infarcted heart wall), and/or contacting medical personnel. The personal alerting device 30 can instruct the patient to perform certain actions, such as to sit quietly while additional diagnostics are performed or to prick his finger for a blood-born biomarker analysis.

The personal alerting device 30 can instruct a bystander rescuer or proximate caregiver to take prescribed actions (e.g., performing cardiopulmonary resuscitation). The personal alerting device 30 can communicate with another diagnostic device or activate a therapeutic device (e.g., an automated external defibrillator located near the patient). The personal alerting device 30 can alert a biomedical technician or the manufacturer of the implanted diagnostic device 10 or the manufacturer of the personal alerting device 10 for device service alarm signals (e.g., the battery is low in either the implanted diagnostic device 10 or the personal alerting device 30).

In the event that the patient does not acknowledge the alarm signal, the personal alerting device 30 can automatically notify emergency medical system 44 or bystander rescuers 46. The personal alerting device 30 can alert an emergency responder through the emergency dispatch system. The personal alerting device 30 can be visible and recognizable to bystanders and emergency medical technicians, so that its information can initiate appropriate resuscitative responses when the patient is unable to respond. The personal alerting device 30, in some embodiments, can provide specific directions to emergency medical technicians or bystander rescuers (e.g., instructions to perform cardiopulmonary resuscitation). In some embodiments, the personal alerting device 30 includes automatic or manual controls for initiating or relaying the alarm signal and associated data to responders, such as via the communication module 36 using short message service communication or the emergency 911 telephone system. The communication module 36 can also be used by proximate or distant rescuers to gain control of the personal alerting device 30 in order to initiate diagnostic functions (e.g., transient biosensors) or therapeutic functions (e.g., drug release) in the implanted diagnostic device 10. In some embodiments, the alerting device can signal its location, for example, via a global positioning system or cell-phone triangulation. The personal alerting device 30 can include suitable security functions so that patient data is not compromised.

The alarm signals can be tailored to different forms according to the type of the alarm and the urgency of the alarm. The personal alerting device 30 can receive data from medical providers to tailor the device for improved performance, for example, by modifying alerting thresholds or updating baseline waveforms for comparison. Upon review of the information stored in the memory 32, the findings of the medical providers can be incorporated as additional event templates that signify positive or negative events, allowing the performance of the personal alerting device 30 to be tailored and improved over time. Further alarm signals can be generated by trends in the patient's data. Authorized medical devices can override functions of the personal alerting device 30 and/or communication channels being used by the communication module 36. Service alarm signals from the implanted diagnostic device 10, such as low battery alarms, can also be transmitted to medical providers or device manufacturers.

In some embodiments, the personal alerting device 30 can include communication and control functions to allow users to manually or automatically acknowledge or silence automatic alarm signals; initiate event signals when symptoms or predefined events occur (e.g., taking medication); relay alarm signals and supporting reports to bystanders (via the display 38), to designated responders (via short message service communication), or to professional caregivers (via an emergency call center or the emergency 911 telephone system).

In some embodiments, the personal alerting device 30 can include communication and control functions to allow responders, caregivers, and authorized medical devices to query the personal alerting device 30 for additional stored data; to gain control of the personal alerting device 30 to initiate diagnostic functions (e.g., transient biosensors); to activate therapeutic functions (e.g., drug release) in the implanted diagnostic device 10; to reset the personal alerting device 30; to override alarm functions; and/or to access device communications channels to send supplemental information to responders and caregivers.

In some embodiments, the personal alerting device 30 can include communication and control functions to allow service personnel to upload parameters, baseline data, and service updates; to change the language of the device; to assess device status and condition; to set network access parameters (e.g., contact numbers and authorization codes); and/or to test communication channel integrity and response times. In some embodiments, the personal alerting device 30 can periodically synchronize clocks with the implanted diagnostic device 10. In some embodiments, a message can be passed between the personal alerting device 30 and the implanted diagnostic device 10 for the purpose of ensuring the personal alerting device 30 is within range. If the personal alerting device 30 is not within range, the personal alerting device 30 can initiate an alarm to warn the user that he needs to keep it closer to his body.

The following paragraphs describe a sequence of events in which the personal alerting device 30 can operate to provide alarm signals during acute myocardial infarctions or other events. When the patient has an acute myocardial infarction event, the implanted diagnostic device 10 can use an alerting algorithm to detect a change in the ST segment. In one embodiment, the implanted diagnostic device 10 or the personal alerting device 30 can determine when an absolute ST deviation exceeds 1 mV for more than five minutes. The implanted diagnostic device 10 generates and sends an event signal to the personal alerting device 30. The personal alerting device 30 begins beeping and/or vibrating and displays an alarm signal to the patient (e.g., a warning message to the patient). In one embodiment, the warning message can indicate the following: “Call 911” and “Potential myocardial infarction detected.” This type of warning message helps to ensure that the emergency medical system 44 is informed correctly of the reason for the 911 call and the gravity of the situation.

The personal alerting device 30 can also display a timestamp of the onset of the ST segment changes, such as “Onset of ST segment changes at 1:35 AM, Jul. 31, 2008.” In some embodiments, the display 38 can show the ST-elevated electrogram signal and a baseline (non-ST-elevated) electrogram signal for the patient. In some embodiments, the personal alerting device 30 can be capable of displaying up to two different messages, depending on the severity of the diagnosis by the implanted diagnostic device 10. The display 38 can include a liquid crystal display screen. However, the display 38 can be as simple as including a blinking light next to text printed directly on the personal alerting device 30. The personal alerting device 30 can continue to display the message for a duration sufficient to be viewed by an emergency medical technician and/or provide the ability to recall the message at a later time.

The personal alerting device 30 can continue beeping until acknowledged via the acknowledge/silence alarm button 42 by the patient. The patient can then call the emergency 911 telephone system and inform the emergency medical system 44 of any symptoms that he or she may be having, as well as the fact that the personal alerting device 30 is conveying the occurrence of a potential acute myocardial infarction. The personal alerting device 30 can transmit (e.g., via cellular telephone service) the alarm signal and the stored diagnostic data to medical personnel. The medical personnel can be a triage nurse at a medical facility or a third-party call center. The medical personnel can call the patient back immediately on the cell phone number that originated the transmission and can establish voice contact with the patient or a caregiver. In one embodiment, the personal alerting device 30 can include an integral cellular phone so that emergency personnel can establish voice contact with the patient. If no one has called the emergency 911 telephone system yet, the medical personnel can also activate the emergency medical system 44 directly.

After the emergency medical technician arrives and perform a 12-lead electrocardiogram, the patient has the option of showing the emergency medical technician the onset timestamp (i.e., the time when the acute myocardial infarction started, the life-threatening trend started, or the patient pushed the patient activator button 40) stored in the personal alerting device 30. If the 12-lead electrocardiogram shows elevation of the ST segment, the patient can be triaged according to the emergency medical system 44 guidelines for that location. The emergency medical technicians will generally take the patient straight to the cardiac care unit if the ST segment is evident on the 12-lead electrocardiogram.

In one embodiment, the personal alerting device 30 can be capable of beeping loudly enough that it is heard within one minute, assuming it is within a two meter proximity of the patient. Similarly, the personal alerting device 30 can be capable of vibrating forcefully enough that it is noticed by the patient within one minute, assuming it is in contact with the patient's body. The audible alarm signal tone or beeping of the personal alerting device 30 can be distinct in perception from device service alarms when the implanted diagnostic device 10 needs service. The audible alarm signal tone or beeping can also be distinct from household items, cell phones, and other patient monitors.

In one embodiment, the personal alerting device 30 can be capable of receiving a wireless telemetry alarm signal from the implanted diagnostic device 10 within two minutes of ST detection, assuming it is within a two meter proximity of the patient. In one embodiment, the implanted diagnostic device 10 can also be capable of sending a wireless telemetry alarm signal for a duration of up to five minutes. The entire memory of the implanted diagnostic device 10 (e.g., greater than approximately 1 megabyte) can be communicated to the personal alerting device 30. In one embodiment, the personal alerting device 30 can be capable of transmitting the entire memory of the implanted diagnostic device 10 and/or the entire memory 32 of the personal alerting device 30 (e.g., greater than approximately 1 megabyte) to a physician programmer within two minutes. The implanted diagnostic device 10 can be capable of storing an electrogram strip of the signal that triggered the acute myocardial infarction alert and a baseline electrocardiogram strip from a predetermined time preceding the acute myocardial infarction for comparison later review via an implanted device programmer or an implanted device monitor.

If the personal alerting device 30 is out of range of the patient and the implanted diagnostic device 10, an alarm signal can begin (e.g., as a backup alarm) and can repeat every few minutes until interrogated by a implanted device programmer or an implanted device monitor, or until the personal alerting device 30 is brought back within range of the implanted diagnostic device 10. The implanted diagnostic device 10 can be capable of producing an audible or vibratory signal every five minutes as a backup alarm until a session begins with an implanted device programmer or an implanted device monitor.

In some embodiments, the implanted diagnostic device 10 can send an alarm signal to a home monitoring system, and the home monitoring system can respond that the event signal has been received. In this manner, the implanted diagnostic device 10 can stop sending the event signal once the signal is received by the home monitoring system (whether or not the alarm signal is received and acknowledged by the personal alerting device 30). In some embodiments, the personal alerting device 30 can capture the event signal and send the device memory to the home monitoring system (which in turn can send the device memory to an internet-based service that allows the physician to see the device information via the internet).

The implanted device programmer or the implanted device monitor can be capable of ignoring a wireless telemetry alarm from the implanted diagnostic device 10 if it is specifically intended for a personal alerting device 30. The personal alerting device 30 can be capable of pairing only with a specific implanted diagnostic device 10. The personal alerting device 30 can have the ability to ignore communications from any implanted device with which it has not been paired.

The acknowledge/silence button 42 can be used to acknowledge the occurrence of an acute myocardial infarction event, and the display 38 of the personal alerting device 30 can provide feedback to the patient that the acknowledgement has been received. The acknowledgement/silence button 42 can also resist accidental acknowledgement.

The personal alerting device 30 can be small enough that it can be carried on the patient at all times. In some embodiments, the personal alerting device 30 can have additional functionality that makes it more desirable for the patient to carry it at all times. The display 38 can show basic status information (e.g., device battery OK, patient OK, current heart rate=85 beats per minute, etc.). The display 38 can show advanced diagnostic data (e.g., fluid accumulation status/trends, pressure trends, etc.). The display 38 can provide exercise monitor-type features, including activity trends, heart rate trends, etc. In some embodiments, the personal alerting device 30 can include cellular phone services. In some embodiments, the personal alerting device 30 can replace an implantable device monitor. In some embodiments, the personal alerting device 30 can operate normally with recharging/battery replacement no more frequently than once per week.

In some embodiments, the personal alerting device 30 can also communicate other non-acute myocardial infarction alarms, such as lead fractures, renal failure, etc.

In some embodiments, the personal alerting device 30 can provide an external pressure reference for an implantable hemodynamic monitor. The implantable hemodynamic monitor can continuously monitor pressure within the heart, body temperature, patient activity, and heart rate in patients with heart failure. The personal alerting device 30 can communicate with the implantable hemodynamic monitor and/or provide alarm functions for the implantable hemodynamic monitor.

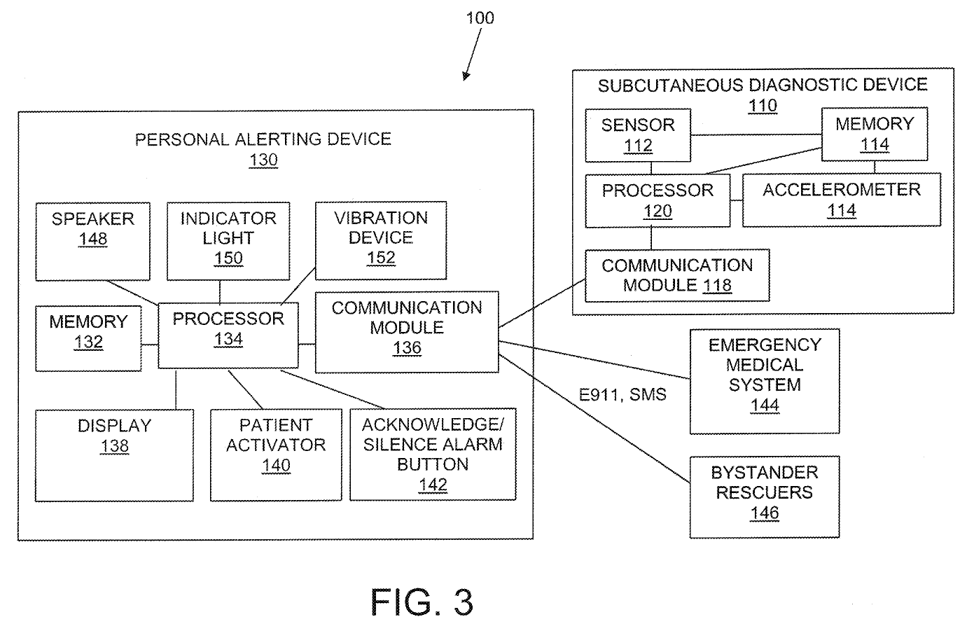

FIG. 3 illustrates a personal alerting system 100 including a subcutaneous diagnostic device 110 and a personal alerting device 130. In one embodiment, the subcutaneous diagnostic device 110 can include a sensor 112 that can obtain a one-lead electrocardiogram. In some embodiments, the subcutaneous diagnostic device 110 can include an accelerometer 114 that can monitor the activity level and position of the patient. For example, the accelerometer 114 can detect if the patient has fallen and is no longer moving. The subcutaneous diagnostic device 110 can include memory 116, a communication module 118, and a processor 120.

The personal alerting device 130 can include memory 132, a processor 134, a communication module 136 (that can communicate with emergency medical system 144 and bystander rescuers 146), a display 138, a patient activator 140, and an acknowledge/silence alarm button 142. The communication module 118 of the subcutaneous diagnostic device 110 can communicate with the communication module 136 of the personal alerting device 130 when the processor 120 determines that the one-lead electrocardiogram is exhibiting an elevated ST segment. The subcutaneous diagnostic device 110 can communicate that an elevated ST segment detection has occurred, along with a timestamp (i.e., when the first elevated ST segment was detected), without necessarily communicating diagnostic data to the personal alerting device 130. The personal alerting device 130 can then perform and include any of the features as described above with respect to FIGS. 1-2.

While the system and method have been described in terms of what are presently considered to be specific embodiments, the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

1. A method of generating an alert of an event detected by an implanted diagnostic device, the method comprising:
providing a personal alerting device;
transmitting an event signal to the personal alerting device from the implanted diagnostic device when at least one of ischemia is detected, a deviation from a baseline electrocardiogram waveform is detected, a life-threatening trend begins, and symptoms are indicated;
storing the event signal in the personal alerting device, the event signal including an event onset timestamp, an event type, and an event electrocardiogram waveform;
generating an alarm signal from the personal alerting device, the alarm signal including at least one of a visual message, a light, a vibration, and a sound; and
prompting further action to be taken through the personal alerting device.

2. The method of claim 1 and further comprising transmitting an event signal from an implanted diagnostic device that is positioned subcutaneously.

3. The method of claim 1 wherein transmitting an event signal to the personal alerting device from the implanted diagnostic device when ischemia is detected includes detecting a new elevated ST segment in the patient's electrocardiogram.

4. The method of claim 1 and further comprising providing a button on the personal alerting device to at least one of acknowledge and silence the alarm signal.

5. The method of claim 1 and further comprising providing a patient activator button on the personal alerting device that allows an event signal to be triggered when symptoms occur.

6. The method of claim 1 and further comprising automatically relaying at least one of the event signal and the alarm signal to an emergency medical system.

7. The method of claim 1 and further comprising initiating at least one of enhanced recording functions and enhanced diagnostic functions in the implanted diagnostic device when the event signal is transmitted to the personal alerting device.

9. The method of claim 1 and further comprising instructing the implanted diagnostic device to perform further diagnostic tests after transmitting the event signal.

10. The method of claim 1 and further comprising initiating therapeutic mitigations after receiving the event signal.

11. The method of claim 1 and further comprising storing patient information in the personal alerting device, the patient information including at least one of contact information, baseline electrocardiogram waveforms, historical data, periodic data, and episode data.

12. The method of claim 1 and further comprising displaying resuscitative response instructions on the personal alerting device.

13. The method of claim 1 and further comprising relaying the event signal via short message service.

14. The method of claim 1 and further comprising tailoring the personal alerting device by at least one of modifying thresholds and updating baseline electrocardiogram waveforms.

15. A personal alerting system that generates an alert of an event, the personal alerting system comprising:
an implanted diagnostic device that transmits an event signal when at least one of ischemia is detected, a deviation from a baseline electrocardiogram waveform occurs, a life-threatening trend begins, and symptoms are indicated;
a patient alerting device including
a processor;
a communication module connected to the processor, the communication module receiving the event signal from the implantable diagnostic device;
memory connected to the processor and the communication module, the memory storing the event signal, the event signal including an event onset timestamp, an event type, and an event electrocardiogram waveform;
at least one of a speaker, an indicator light, and a vibration generator that generates a perceptible alarm signal when the communication module receives an event signal; and
a display connected to the processor and the memory, the display indicating the alarm signal and prompting further action to be taken.

16. The personal alerting device of claim 15 wherein the communication module receives an event signal when the implanted diagnostic device detects a new elevated ST segment in an electrocardiogram.

17. The personal alerting device of claim 15 and further comprising a button to at least one of acknowledge and silence the alarm signal.

18. The personal alerting device of claim 15 and further comprising a patient activator button that allows an event signal to be triggered when symptoms occur.

19. The personal alerting device of claim 15 wherein the memory stores patient information in the personal alerting device, the patient information including at least one of contact information, baseline electrocardiogram waveforms, historical data, periodic data, and episode data.

20. A method of generating an alert of an event detected by an implanted diagnostic device, the method comprising:
providing a personal alerting device;
transmitting an event signal to the personal alerting device from the implanted diagnostic device when ischemia is detected;
storing the event signal in the personal alerting device, the event signal including an event onset timestamp, an event type, and an event electrocardiogram waveform; and
generating an alarm signal from the personal alerting device, the alarm signal including at least one of a visual message, a light, a vibration, and a sound.

21. The method of claim 20 and further comprising transmitting an event signal from one of an implanted diagnostic device and a subcutaneous diagnostic device.

22. The method of claim 20 wherein transmitting an event signal to the personal alerting device from the implanted diagnostic device when ischemia is detected includes detecting a new elevated ST segment in an electrocardiogram.

23. The method of claim 20 and further comprising initiating at least one of enhanced recording functions and enhanced diagnostic functions in the implanted diagnostic device when the event signal is transmitted to the personal alerting device.

24. The method of claim 20 and further comprising instructing the implanted diagnostic device to perform further diagnostic tests after transmitting the event signal.

25. The method of claim 20 and further comprising storing patient information in the personal alerting device, the patient information including at least one of contact information, baseline electrocardiogram waveforms, historical data, periodic data, and episode data.

26. The method of claim 20 and further comprising tailoring the personal alerting device by at least one of modifying thresholds and updating baseline electrocardiogram waveforms.

27. A method of alerting medical personnel of an event being experienced by a patient, the method comprising:
providing an implanted diagnostic device and a personal alerting device;
transmitting an event signal to the personal alerting device from the implanted diagnostic device when ischemia is detected;
generating an alarm signal indicating that an acute myocardial infarction has been detected;
automatically contacting the medical personnel when the event signal is received; and
establishing voice communication between the medical personnel and the patient.

28. The method of claim 27 and further comprising screening the event signal for false positive alarms before alerting an emergency medical system.

29. The method of claim 27 wherein the personal alerting device forwards the event signal and accompanying data to the medical personnel.

30. The method of claim 27 wherein the implanted diagnostic device detects ischemia.

31. The method of claim 27 and further comprising displaying on the patient alerting device in substantially real time at least one of an electrocardiogram, an electrogram, blood pressure, oxygen, cardiac output, and heart contractility.

32. The method of claim 27 and further comprising displaying on the patient alerting device instructions to administer at least one of aspirin and nitroglycerin.

33. The method of claim 27 and further comprising automatically contacting the medical personnel if the alarm signal is not acknowledged within a predetermined time period.

34. The method of claim 27 and further comprising providing a voice-activated system in the personal alerting device that allows the patient to at least one of indicate he is experiencing symptoms, initiate additional diagnostic data collection, and communicate with medical personnel.

35. The method of claim 27 wherein the personal alerting device performs a signal quality check on the event signal before generating the alarm signal.

36. The method of claim 27 wherein the personal alerting device records a surface electrocardiogram after receiving the event signal.

37. The method of claim 27 wherein the personal alerting device stores data corresponding to the event signal including at least one of an onset timestamp, an event type, a duration, an amplitude, an event electrocardiogram, an ST deviation value, a baseline electrocardiogram, and an electrogram.

38. The method of claim 27 wherein the personal alerting device stores a digitized baseline twelve-lead electrocardiogram.

39. The method of claim 27 wherein the personal alerting device analyzes additional diagnostic data after receiving the event signal and before generating an alarm signal.

40. The method of claim 39 wherein the additional diagnostic data includes a change in blood pressure.

41. The method of claim 27 wherein the personal alerting device communicates with an automated external defibrillator.

42. The method of claim 27 wherein the personal alerting device generates device service alarms for at least one of the implanted diagnostic device and the personal alerting device.

43. The method of claim 27 wherein the medical personnel communicate with the personal alerting device in order to at least one of obtain stored data, initiate diagnostic functions, initiate therapeutic functions, modify alert thresholds, and update a baseline electrocardiogram.

44. The method of claim 43 wherein the therapeutic functions include drug release.

45. The method of claim 27 wherein the personal alerting device signals its location to the medical personnel via at least one of a global positioning system and cellular phone triangulation.
